(19) United States
(12) Patent Application Publication
(10) Pub. No.: US 2023/0125732 A1
(43) Pub. Date: Apr. 27, 2023

(54) SYSTEMS AND METHODS FOR PRODUCING MIXTURES

(71) Applicants: Boston Scientific Medical Device Limited, Galway (IE); Boston Scientific Scimed, Inc., Maple Grove, MN (US)

(72) Inventors: Benjamin CLEVELAND, Bellingham, MA (US); Nitesh Ghananil BAVISKAR, Kalyan West (IN); Junaid Mohammed SHAIKH, Surat (IN); Richard Earl GRAFFAM, Pelham, NH (US); Joseph HERNANDEZ, Rutland, MA (US); Christopher WATSON, Lincoln, MA (US); Jennie CREEGAN, Minneapolis, MN (US); Kolbein KOLSTE, Acton, MA (US); Nicholas DeSANTIS, Cambridge, MA (US); Eric KIM, Brighton, MA (US); Mei Lee AMEND, Lakeville, MA (US)

(73) Assignees: Boston Scientific Medical Device Limited, Galway (IE); Boston Scientific Scimed, Inc., Maple Grove, MN (US)

(21) Appl. No.: 18/048,571

(22) Filed: Oct. 21, 2022

Related U.S. Application Data
(60) Provisional application No. 63/262,949, filed on Oct. 22, 2021.

(51) Int. Cl. A61J 1/20

(52) U.S. Cl. CPC A61J 1/201; A61J 1/202; A61J 1/2089; A61J 1/2068

(57) ABSTRACT

A system is disclosed for producing a mixture to deliver to a treatment site. The system includes a mixing lumen attachable to a proximal end of a delivery system. A multi-lumen chamber can be removably connected to a proximal end of the mixing lumen and include a first lumen aligned with a second lumen. The first lumen can be configured to comprise a first constituent in a first state. The first lumen can include a first plunger to move the first constituent from the first lumen to the mixing lumen. The first lumen can terminate in a first port. The second lumen can be configured to include a second constituent. The second lumen can include a second plunger to distally move the second constituent and terminate in a second port. A vial adaptor can be included with a vial having a third constituent.

### CROSS-REFERENCE TO RELATED APPLICATIONS

This patent application claims the benefit of priority under 35 U.S.C. § 119 to U.S. Provisional Patent Application No. 63/262,949, filed Oct. 22, 2021, which is herein incorporated by reference in its entirety.

### FIELD

The present disclosure relates generally to compositions for injection to a patient, methods of preparation and use thereof, and devices comprising such compositions.

### BACKGROUND

Numerous men are diagnosed with prostate cancer each year. Traditionally, treatment options include interstitial implant therapy, surgery, and external beam radiotherapy. While the best treatment is still debatable, side effects of treating prostate cancer have become less toxic with implant therapy and radiotherapy.

Since the conception of conformal radiotherapy, physicians have paid attention to the delivered dose to the target and surrounding tissues. Investigators have been able to correlate side effects to the amount of tissue receiving a certain radiation dose. And yet, time, distance, and shielding affect the dose that is delivered. The less time an area is exposed to radiation, the less dose delivered. The greater the distance from the radiation, the less dose delivered.

Current systems provide filler material to treatment sites to decrease the radiation dose to the rectum during radiotherapy for prostate cancer. However, the system that mixes the filler material in vitro includes numerous subcomponents, is complex to assemble, and rife with filler mixing errors prior to delivery within a patient at a treatment site. During the foregoing procedures, such errors and mishaps lead unnecessarily to patient risk, increased procedure time, and increased procedure costs. The solution of this disclosure resolves these and other issues of the art.

### SUMMARY

In accordance with certain embodiments of the present disclosure, a system is disclosed for producing a mixture to deliver to a treatment site. The system can include a mixing lumen attachable to a proximal end of a delivery system. A multi-lumen chamber can be removably connected to a proximal end of the mixing lumen. The multi-lumen chamber can include a first lumen aligned with a second lumen. The first lumen can be configured to include a first constituent in a first state, the first lumen including a first plunger to move the first constituent from the first lumen to the mixing lumen. The first lumen can terminate in a first port. The second lumen can be configured to include a second constituent. The second lumen can include a second plunger to distally move the second constituent. The second lumen can terminate in a second port. A vial adaptor can be included with a vial that includes a third constituent, the vial adaptor being configured to connect to the multi-lumen chamber.

In accordance with certain embodiments of the present disclosure, the first and second lumens are adjacent each other.

In accordance with certain embodiments of the present disclosure, the second lumen can include a second plunger rod internally positioned therein to distally move the second constituent and the first constituent.

In accordance with certain embodiments of the present disclosure, in a third state, distally moving the second plunger rod causes a first mixture and the second constituent to be delivered through the first and second ports, mixed together within the mixing lumen to form the mixture, and delivered through the delivery system.

In accordance with certain embodiments of the present disclosure, in a second state, proximally moving the first plunger causes a first mixture including the first and third constituents to be transported from the vial to the first lumen.

In accordance with certain embodiments of the present disclosure, the mixing lumen is in a connector that includes a first asymmetric coupler positioned on a proximal end of the connector.

In accordance with certain embodiments of the present disclosure, the multi-lumen chamber includes a second asymmetric coupler positioned on a distal end of the multi-lumen chamber and is configured to removably connect to the first asymmetric coupler.

In accordance with certain embodiments of the present disclosure, the vial adaptor includes an asymmetric coupler including an outer profile substantially similar to the first asymmetric coupler so as to removably connect with the second asymmetric coupler.

In accordance with certain embodiments of the present disclosure, in the first state, the second asymmetric coupler is connected with the asymmetric coupler of the vial adaptor so that distally moving a first plunger rod of the first lumen causes the first constituent to be delivered through the first port into the vial to form a first mixture.

In accordance with certain embodiments of the present disclosure, the vial adaptor further includes a first fluid port to removably connect with the first port and a second alignment port to removably connect with the second port, the first fluid port and the second alignment port being surrounded by the outer profile of the asymmetric coupler.

In accordance with certain embodiments of the present disclosure, the asymmetric coupler of the vial adaptor includes a vial receiver with a plurality of radially separated tabs that collectively form a flexible inner diameter to removably connect to an outer diameter of the vial.

In accordance with certain embodiments of the present disclosure, the outer profile of the asymmetric coupler is shaped so that the first port can only removably connect with the first fluid port and the second port can only removably connect with the second alignment port.

In accordance with certain embodiments of the present disclosure, the second alignment port includes an outer diameter configured to be inserted into the second port. The second alignment port can include a dowel configured to plug the second port and prevent flow of the second constituent.

In accordance with certain embodiments of the present disclosure, the vial adaptor can include a tube between the vial and the first fluid port. A vent can be included for venting air in the tube, the vial, and the first fluid port, the vent being positioned between the vial and the first fluid port.

In accordance with certain embodiments of the present disclosure, the tube includes one or more approximately perpendicular bends distal of the vial and proximal of the first fluid port. The vent can be positioned proximal or adjacent the one or more approximately perpendicular bends.

In accordance with certain embodiments of the present disclosure, the vent includes a one-way valve including an air-permeable fluid-impermeable membrane.

In accordance with certain embodiments of the present disclosure, the asymmetric coupler of the vial adaptor includes one or more latches configured to removably connect with one or more latches of the first asymmetric coupler. The asymmetric coupler can include an actuator positioned on the outer profile configured to be squeezed so as to release a coupling between the one or more latches of the first asymmetric coupler and the asymmetric coupler.

In accordance with certain embodiments of the present disclosure, a method is disclosed for producing a mixture with a mixing system to deliver to a treatment site. The mixing system can include a multi-lumen chamber including a first lumen aligned with a second lumen and an asymmetric coupler on a distal end of the multi-lumen chamber. The first lumen can include a first constituent and a first plunger rod internally positioned to move one or more constituents from the first lumen through a first port positioned at a distal end. The second lumen can include a second constituent and a second plunger rod internally positioned to move one or more constituents from the first and second lumens lumen through the first port and a second port positioned at a distal end of the second lumen. The method can include connecting an asymmetric coupler of a vial adaptor to the asymmetric coupler of a mixing system, the vial adaptor including a vial with a third constituent; distally moving the first plunger rod causing the first constituent to be delivered through the first port into the vial to mix with the third constituent and form a first mixture; proximally moving the first plunger rod causing the first mixture to be transported from the vial to the first lumen; detaching the vial adaptor from the mixing system; connecting an asymmetric coupler of a connector to the asymmetric coupler of the mixing system, the connector including a central lumen attached to a proximal end of a needle; and distally moving the second plunger rod causing the first mixture and the second constituent to be delivered through the first and second ports and mixed together within the central lumen of the connector to form the mixture.

In accordance with certain embodiments of the present disclosure, the vial adaptor can include a first fluid port to removably connect with the first port and a second alignment port to removably connect with the second port, the first fluid port and the second alignment port being surrounding by an outer profile of the asymmetric coupler.

In accordance with certain embodiments of the present disclosure, the asymmetric coupler of the vial adaptor includes a vial receiver with a plurality of radially separated tabs that collectively form a flexible inner diameter to removably connect to an outer diameter of the vial.

In accordance with certain embodiments of the present disclosure, the vial adaptor further includes a first fluid port to removably connect with the first port and a second alignment port to removably connect with the second port, wherein an outer profile of the asymmetric coupler is shaped so that the first port can only removably connect with the first fluid port and the second port can only removably connect with the second alignment port.

In accordance with certain embodiments of the present disclosure, the vial adaptor further includes a first fluid port to removably connect with the first port and a second alignment port to removably connect with the second port, the second alignment port including an outer diameter configured to be inserted into the second port, the second alignment port including a dowel configured to plug the second port and prevent flow of the second constituent.

In accordance with certain embodiments of the present disclosure, the vial adaptor further includes a first fluid port to removably connect with the first port and a second alignment port to removably connect with the second port. A tube can be between the vial and the first fluid port and a vent for venting air in the tube, the vial, and the first fluid port. The vent can be positioned between the vial and the first fluid port.

In accordance with certain embodiments of the present disclosure, the tube can include one or more approximately perpendicular bends distal of the vial and proximal of the first fluid port. The vent can be positioned proximal or adjacent the one or more approximately perpendicular bends.

In accordance with certain embodiments of the present disclosure, the asymmetric coupler of the vial adaptor can include one or more latches configured to removably connect with one or more latches of the first asymmetric coupler. The asymmetric coupler can include an actuator positioned on an outer profile configured to be squeezed so as to release a coupling between the one or more latches of the first asymmetric coupler and the asymmetric coupler.

In accordance with certain embodiments of the present disclosure, the method can include purging air from the first and second lumens by distally moving the second plunger rod a first distance during delivery of the gel composition.

In accordance with certain embodiments of the present disclosure, the connector is a Y-shaped connector and the method can include positioning the asymmetric coupler at or adjacent the connector; positioning a first tube in fluid communication with the first fluid port and the central lumen of the connector; and positioning a second tube in fluid communication with the second port and the central lumen of the connector

To the accomplishment of the foregoing and related ends, certain illustrative aspects are described herein in connection with the following description and the appended drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE FIGURES

The accompanying drawings, which are incorporated in and constitute a part of this specification, illustrate various exemplary aspects of the disclosure, and together with the description serve to explain the principles of the present disclosure.

FIGS. 1A-1B depict the prostate, rectum, and Denonvilliers' space between the prostate and rectum.

FIG. 2 shows an exploded view of an exemplary mixing system in accordance with certain aspects of the present disclosure.

FIG. 3 depicts a plunger assembly in accordance with certain aspects of the present disclosure.

FIG. 4 depicts a partial cross-section view of an example vial and attached to an example mixing system, in accordance with certain aspects of the present disclosure.

FIG. 5A depicts an example step in a method using an example mixing system, in accordance with certain aspects of the present disclosure.

FIG. 5B depicts an example step in a method using an example mixing system, in accordance with certain aspects of the present disclosure.

FIG. 6A depicts an example step in a method using an example mixing system, in accordance with certain aspects of the present disclosure.

FIG. 6B depicts an example step in a method using an example mixing system, in accordance with certain aspects of the present disclosure.

FIG. 7A depicts an example step in a method using an example mixing system, in accordance with certain aspects of the present disclosure.

FIG. 7B depicts an example step in a method using an example mixing system, in accordance with certain aspects of the present disclosure.

FIG. 8A depicts an example step in a method using an example mixing system, in accordance with certain aspects of the present disclosure.

FIG. 8B depicts an example step in a method using an example mixing system, in accordance with certain aspects of the present disclosure.

FIG. 9A depicts an example step in a method using an example mixing system, in accordance with certain aspects of the present disclosure.

FIG. 9B depicts an example step in a method using an example mixing system, in accordance with certain aspects of the present disclosure.

FIGS. 10A-10C depict example steps in a method of priming an example connector, in accordance with certain aspects of the present disclosure.

FIG. 11 depicts a flow diagram of a method of using a mixing system according to certain aspects of this disclosure.

### DETAILED DESCRIPTION

Particular aspects of the present disclosure are described in greater detail below. The terms and definitions provided herein control, if in conflict with terms and/or definitions incorporated by reference.

Particular aspects of the present disclosure may repeat reference numerals and/or letters in the various examples. This repetition is for the purpose of simplicity and clarity and does not in itself dictate a relationship between the various embodiments and/or configurations discussed. Different embodiments may have different advantages, and no particular advantage is necessarily required of any embodiment.

As used herein, the terms “comprises,” “comprising,” or any other variation thereof are intended to cover a non-exclusive inclusion, such that a process, method, composition, article, or apparatus that comprises a list of elements does not include only those elements, but may include other elements not expressly listed or inherent to such process, method, composition, article, or apparatus. The term “exemplary” is used in the sense of “example” rather than “ideal.”

As used herein, the singular forms “a,” “an,” and “the” include plural reference unless the context dictates otherwise.

As used herein, “approximately” and “about” refer to being nearly the same as a referenced number or value. As used herein, the terms “approximately” and “about” should be understood to encompass ±10% of a specified amount or value (e.g., “about 90%” can refer to the range of values from 81% to 99%).

As used herein, “operator” can include a doctor, surgeon, or any other individual or delivery instrumentation associated with delivery or use of a mixing system as such systems are described throughout this disclosure.

The compositions herein may be used in various medical procedures, including but not limited to injected to create additional space between the rectum and prostate during treatment, for example in the Denonvilliers' space, thereby reducing rectal radiation dose and associated side effects. Certain embodiments of the disclosure include placing a filler between the radiation target tissue and other tissues. The filler can be a gel composition that increases the distance between the target tissue and other tissues so that the other tissues receive less radiation.

It is understood that “Denonvilliers' space” is a region located between the rectum and prostate. Certain embodiments provide a method of displacing a tissue to protect the tissue against the effects of a treatment involving radiation or cryotherapy. One embodiment involves using a filler mixed by a mixing system of this disclosure to displace the tissue relative to a tissue that is to receive the treatment. Another embodiment involves introducing a filler mixed by a mixing system of this disclosure to displace a first tissue and radiating a second tissue, particularly a second tissue that is close to the first tissue. In another embodiment, the method includes the steps of injecting a filler into a space between tissues; and may further include irradiating one of the tissues so that the other tissue receives less radiation than it would have in the absence of the filler.

Certain embodiments also provide methods for treating a tissue of a body by radiation. In one embodiment, the method includes the steps of injecting an effective amount of a filler into a space between a first tissue (e.g., prostate) of a body and a second tissue (e.g., rectum), which can be a critically sensitive organ; and treating the first tissue by radiation whereby the filler within the space reduces passage of radiation into the second tissue. Tissue is a broad term that encompasses a portion of a body: for example, a group of cells, a group of cells and interstitial matter, an organ, a portion of an organ, or an anatomical portion of a body, e.g., a rectum, ovary, prostate, nerve, cartilage, bone, brain, or portion thereof.

The gel of the filler can include polymeric materials which are capable of forming a hydrogel may be utilized. In one embodiment, the polymer forms a hydrogel within the body. A hydrogel is defined as a substance formed when an organic polymer (natural or synthetic) is cross-linked via covalent, ionic, or hydrogen bonds to create a three-dimensional open-lattice structure which entraps water molecules to a gel. Naturally occurring and synthetic hydrogel forming polymers, polymer mixtures, and copolymers may be utilized as hydrogel precursors.

In some aspects, the hydrogel can be formed by a composition formed by constituents (e.g., mixing accelerant fluid, diluent, and PEG together) and may include one or more polysaccharide compounds or a salt thereof. For example, the composition may include a cellulose compound such as carboxymethyl cellulose (CMC) or salt thereof (e.g., CMC) sodium, xanthan gum, alginate or a salt thereof (e.g., calcium alginate, such as Ca-alginate beads), chitosan, and/or hyaluronic acid. In some examples, the composition may comprise a mixture of hyaluronic acid and CMC, and/or may be cross-linked with a suitable crosslinking compound, such as butanediol diglycidyl ether (BDDE). In some aspects, the polysaccharide may be a homopolysaccharide or a heteropolysaccharide

The present disclosure also provides mixing systems to form the gel composition and corresponding medical devices for use and/or delivery to a treatment site of a patient. According to some aspects of the present disclosure, the mixing system may include a plurality of reservoirs with respective lumens. Collectively, the lumens therein may serve as a container for constituents to mix the gel composition of this disclosure. Suitable reservoirs may include, for example, syringes (e.g., a syringe barrel compatible with a manual or automatic injection system) and other fluid containers configured for use with a suitable injection needle. Exemplary materials suitable for the reservoir include, but are not limited to, cyclic olefin polymer, polypropylene, polycarbonate, polyvinyl chloride, and glass. In some aspects, one of these materials (e.g., cyclic olefin copolymer specifically) can have a coating applied to it, such as SiO₂), which is advantageous so the coating can perform as a primary oxygen barrier, behave as a glass-like layer, and can be applied using a vapor deposition process.

According to some aspects of the present disclosure, the compositions may include at least one accelerant (e.g., an activating agent) combined with a precursor mixed from a diluent (e.g., mostly water) and polyethylene glycol (PEG). In some examples, the composition may be or include a gel with a desired gel strength and/or viscosity, such as a biocompatible gel suitable for injection (e.g., through a needle).

The hydrophilic polymer can be any gelling agent(s), including natural ones or synthetic in origin, and may be anionic, cationic, or neutral. Non-limiting examples of the gelling agents include polysaccharides such as gellan gum, xanthan gum, gum arabic, guar gum, locust bean gum, alginate, and carrageenans.

The concentrations of gelling agent(s) in the composition described in this disclosure may range from about 0.01% to about 2.0% by weight with respect to the total weight of the composition, such as from about 0.02% to about 1.5%, from about 0.05% to about 1.0%, from about 0.05% to about 0.50%, from 0.05% to about 0.15%, from about 0.10% to about 0.20%, from about 0.15% to about 0.25%, from about 0.20% to about 0.30%, from about 0.25% to about 0.35%, from about 0.30% to about 0.40%, from about 0.35% to about 0.45%, from about 0.40% to about 0.50%, from about 0.1% to about 0.5%, or from about 0.1% to about 0.15% by weight with respect to the total weight of the composition. In at least one example, the total concentration of the gelling agent(s) in the composition may range from about 0.05% to about 0.5% by weight with respect to the total weight of the composition.

In some examples, the composition may have a viscosity ranging from about 0.001 Pascal-second (Pas) to about 0.100 Pa·s at a shear rate of 130 s⁻¹, such as, e.g., from about 0.005 Pa·s to about 0.050 Pa·s, from about 0.010 Pa·s to about 0.050 Pas, from about 0.010 Pa·s to about 0.030 Pa·s, from about 0.010 Pa·s to about 0.020 Pas, from about 0.020 Pa·s to about 0.030 Pa·s, or from about 0.020 Pa·s to about 0.040 Pas at a shear rate of 130 s⁻¹. Thus, for example, the composition may be or comprise a gel having a viscosity of about 0.005 Pa·s, about 0.006 Pa·s, 0.008 Pa·s, about 0.010 Pas, about 0.011 Pa·s, about 0.012 Pa·s, about 0.013 Pa·s, about 0.014 Pa·s, about 0.015 Pa·s, about 0.016 Pa·s, about 0.017 Pa·s, about 0.018 Pa·s, about 0.019 Pa·s, about 0.020 Pa·s, about 0.022 Pa·s, about 0.024 Pa·s, about 0.026 Pa·s, about 0.028 Pas, about 0.030 Pa·s, about 0.032 Pa·s, about 0.034 Pa·s, about 0.036 Pa·s, about 0.038 Pa·s, about 0.040 Pa·s, about 0.042 Pa·s, about 0.044 Pa·s, about 0.046 Pa·s, about 0.048 Pa·s, or about 0.050 Pa·s at a shear rate of 130 s⁻¹. In at least one example, the composition may have a viscosity greater than 0.0050 Pa·s at a shear rate of 130 s⁻¹, e.g., a viscosity ranging from about 0.005 Pa·s to about 0.050 Pa·s, at a shear rate of 130 s⁻¹. In at least one example, the composition may have a viscosity greater than 0.010 Pas at a shear rate of 130 s⁻¹, e.g., a viscosity ranging from about 0.010 Pa·s to about 0.030 Pa·s, at a shear rate of 130 s⁻¹.

Alternatively or additionally, the composition may have a viscosity ranging from about 0.001 Pa·s to about 0.050 Pa·s at a shear rate of 768 s⁻¹, such as, e.g., from about 0.002 Pa·s to about 0.030 Pa·s, from about 0.003 Pa·s to about 0.020 Pa·s, from about 0.004 Pa·s to about 0.010 Pa·s, from about 0.004 Pa·s to about 0.006 Pa·s, from about 0.005 Pa·s to about 0.007 Pa·s, from about 0.006 Pa·s to about 0.008 Pa·s, from about 0.007 Pa·s to about 0.009 Pa·s, or from about 0.008 Pa·s to about 0.01 Pa·s at a shear rate of 768 s⁻¹. Thus, for example, the composition may be or comprise a gel having a viscosity of about 0.003 Pa·s, about 0.004 Pa·s, about 0.005 Pa·s, about 0.006 Pa·s, about 0.007 Pa·s, about 0.008 Pa·s, about 0.009 Pa·s, or about 0.010 Pa·s at a shear rate of 768 s⁻¹. In at least one example, the composition may have a viscosity less than 0.010 Pa·s at a shear rate of 768 s⁻¹, e.g., a viscosity ranging from about 0.005 Pa·s to about 0.009 Pa·s at a shear rate of 768 s⁻¹. In at least one example, the composition may have a viscosity ranging from about 0.004 Pa·s to about 0.010 Pa·s at a shear rate of 768 s⁻¹. Further, for example, the composition may have a viscosity ranging from about 0.010 Pas to about 0.030 Pa·s, e.g., about 0.017 Pa·s at a shear rate of 130 s⁻¹ and a viscosity ranging from about 0.004 Pa·s to about 0.010 Pa·s, e.g., about 0.007 Pa·s, at a shear rate of 768 s⁻¹.

The mixing system herein may include or be removably connected to one or more needles. In some examples, the needle may be a hypodermic needle, and may range from a size of 7 gauge (4.57 mm outer diameter (OD), 3.81 mm inner diameter (ID)) to 33 gauge (0.18 mm OD, 0.08 mm ID), e.g., a size of 16 gauge (1.65 mm OD, 1.19 mm ID), 18 gauge, 21 gauge (0.82 mm OD, 0.51 mm ID), 22 gauge (0.72 mm OD, 0.41 mm ID), 23 gauge (0.64 mm OD, 0.33 ID), or 24 gauge (0.57 mm OD, 0.31 mm ID). Exemplary materials for the needle include, but are not limited to, metals and metal alloys, such as stainless steel and Nitinol, and polymers. The distal tip of the needle may be sharpened, and may have a beveled shape. The proximal end of the needle may include a suitable fitting/adaptor (e.g., a Luer adapter) for engagement with a syringe or other reservoir. In some examples, the needle may include an elongated tube or catheter between the needle tip and the proximal fitting/adapter.

According to some aspects of the present disclosure, the filler compositions herein, e.g., the compositions prepared by the methods herein may have sufficient strength, e.g., gel strength, to withstand the forces and thus minimizing the effects of the forces on the continuity of the three-dimensional gel network. In the meantime, the composition with sufficient strength may have a viscosity suitable for injection, e.g., a viscosity that does not render the composition stuck in the reservoir(s), delivery lumen, or a needle connected therewith.

According to some aspects of the present disclosure, the composition may maintain its three-dimensional structure until the gel is injected through a needle, whereupon the structure may form fragments of the original continuous, three-dimensional network. Those gel fragments may have a diameter corresponding to the diameter of the injection needle, such that the fragments are as large as possible in-vivo to retain as much of the three-dimensional structure of the gel as possible. Injection of these larger-sized particles or fragments is believed to increase the amount of time the gel remains within the tissue.

The amount of force required to move the composition through a needle aperture (generally described as “peak load” force) may depend on the viscosity of the composition, the dimensions of the needle (inner diameter, outer diameter, and/or length), and/or the material(s) from which the needle is formed. For example, a greater amount of force may be applied to inject the composition through a 33-gauge needle in comparison to a 7-gauge needle. Additional factors that may affect the amount of force applied to inject the composition may include the dimensions of a catheter (inner diameter, outer diameter, and/or length) connecting the mixing system to the needle. Suitable peak loads for injection with one or two hands may range from about 5 lbf to about 25 lbf, such as from about 10 lbf to about 20 lbf, e.g., about 15 lbf. The loads measured for a given gel concentration may vary for different needles and flow rates.

According to some aspects of the present disclosure, the size of the needle may be chosen based on the viscosity and/or components of the composition, or vice versa. According to some aspects of the present disclosure, the size of the needle may be 23 gauge or 25 gauge. In some cases, a larger size of 18-gauge, 20 gauge, 21 gauge, or 22 gauge may be used to inject the compositions herein.

According to some aspects of the present disclosure, the mixing system of this disclosure can be included in a kit for introducing a filler into a patient, whereby the filler can include any of the gel compositions of this disclosure. Kits or systems for mixing a gel composition of this disclosure, such as hydrogels, may be prepared so that the precursor(s) and any related activating agent(s) are stored in the kit with diluents as may be needed. Applicators may be used in combination with the same. The kits can be manufactured using medically acceptable conditions and contain components that have sterility, purity and preparation that is pharmaceutically acceptable. Solvents/solutions may be provided in the kit or separately. The kit may include syringes and/or needles for mixing and/or delivery. The kit or system may comprise components set forth herein.

During some examples of use, once saline has been injected to the treatment site, a mixing system can be connected to a needle (e.g., an 18-gauge spinal needle) to then inject a 5-10 mm layer of filler (e.g., gel composition) along the posterior wall of the prostate between the prostate and rectum. Once the filler has been injected into the space between the rectum and prostate, ultrasound images can be obtained.

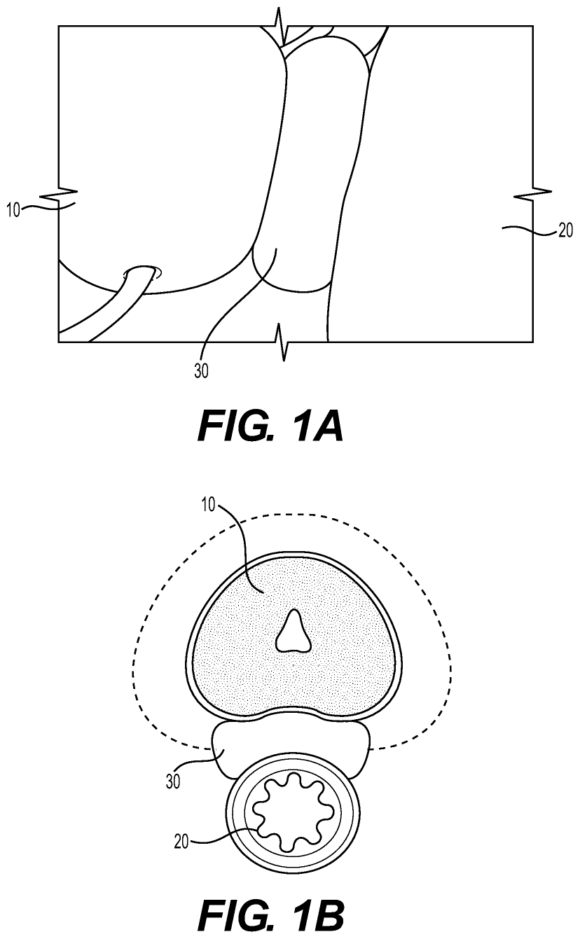

Turning to the drawings, FIG. 1A is a perspective view and FIG. 1B is a partial cross-section view illustrating example filler 30, in the form of a gel composition having been delivered by the mixing system of this disclosure between rectum 20 and prostate 10 of a patient in Denonvilliers' space.

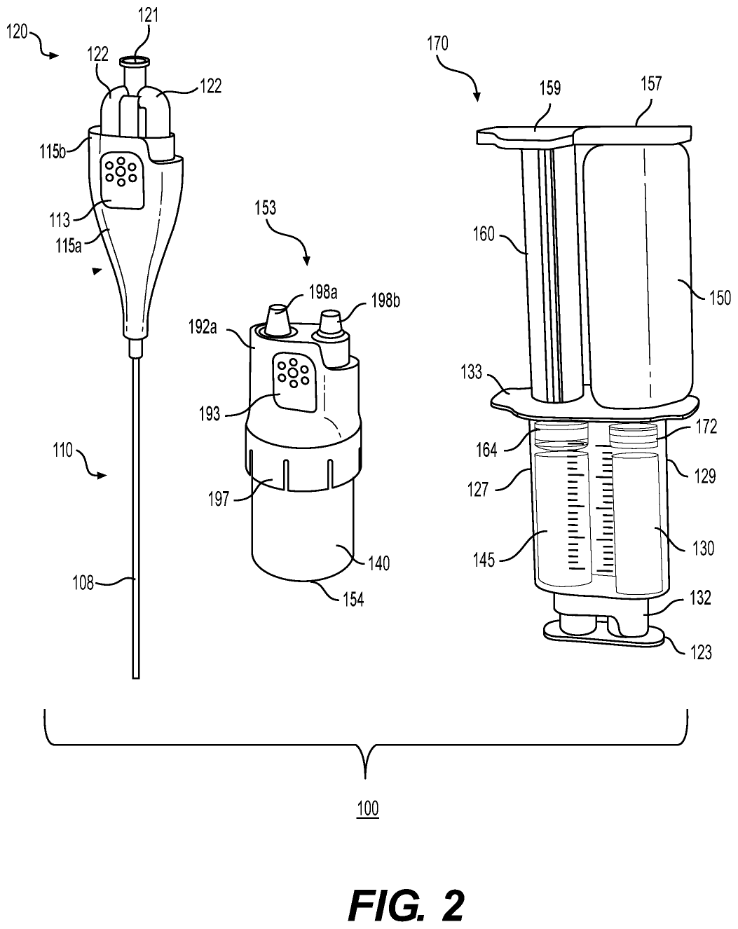

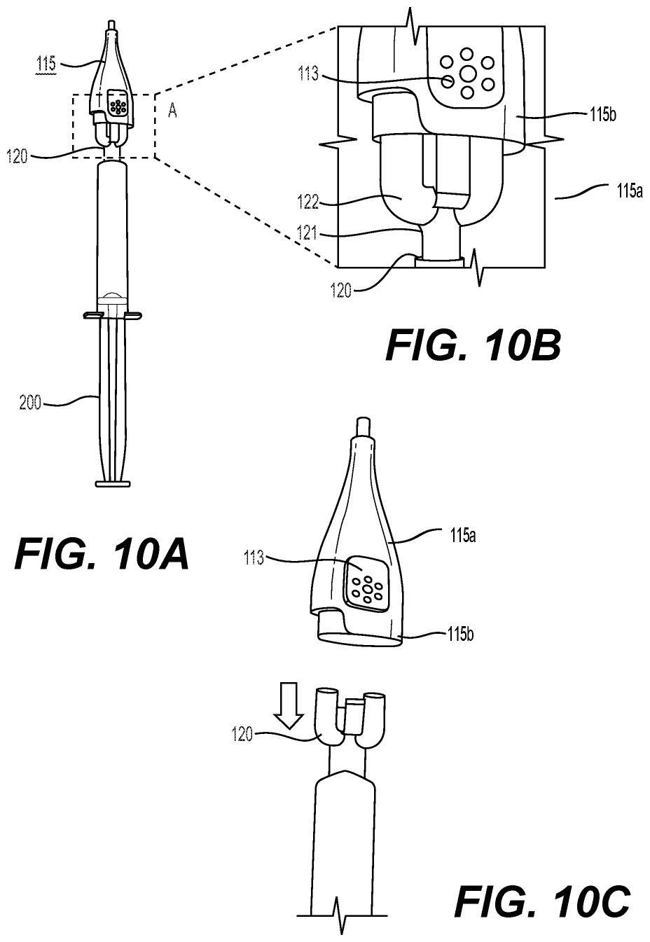

FIG. 2 shows an exploded view of an exemplary mixing system 100 in accordance with certain aspects of the present disclosure for mixing a gel composition for use as filler 30. The system 100 can be packaged in a kit and include a vial adaptor 153 attachable to a main assembly 170 and a needle assembly 110 attachable to the main assembly 170. Needle assembly 110 can include needle 108, which can be any needle of this disclosure suitable for hydrodissection as well as delivering the gel composition (e.g., filler 30) to the treatment site. Connector 115 can also connect to a syringe adaptor 120 for hydrodissection. After hydrodissection with syringe 200, adaptor 120 can be released from connector 115 so that connector can then be connected to assembly 170, as shown in FIGS. 10A-10C. A proximal end of needle 108 can be connected to a distal end of a connector 115 with distal, symmetric portion 115a. Connector 115 can include an asymmetric coupling portion 115b.

Main assembly 170 of system 100 can include a multi-lumen chamber formed by a first lumen 127 inside a first barrel and a second lumen 129 inside a second barrel. Each lumen 127, 129 can be oriented parallel with the other, running side-by-side. A plunger stopper 164 can be located at a distal end of a first plunger rod 160. Rod 160 can be advanceable within lumen 127. Rod 160 can be advanced by flange 159 positioned on a proximal end of rod 160. In some examples, flange 159 is shaped and arranged so that it is prevented from advancing proximally past flange 157.

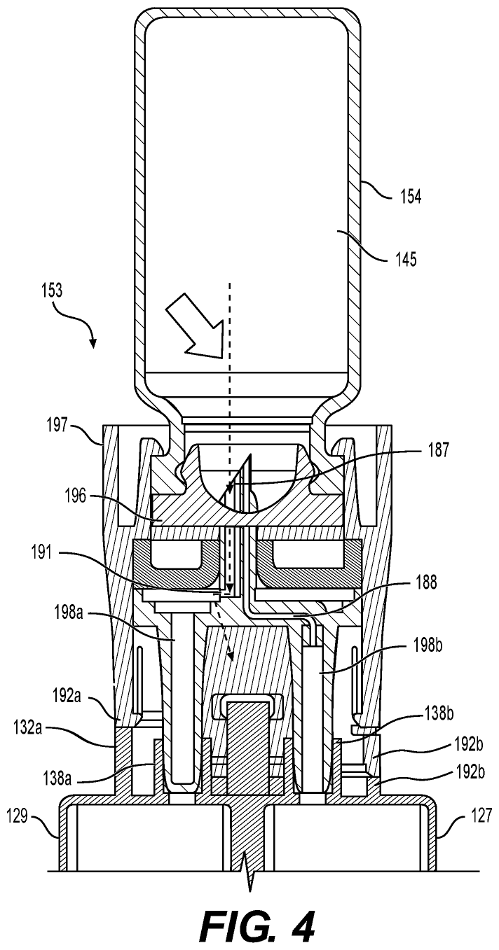

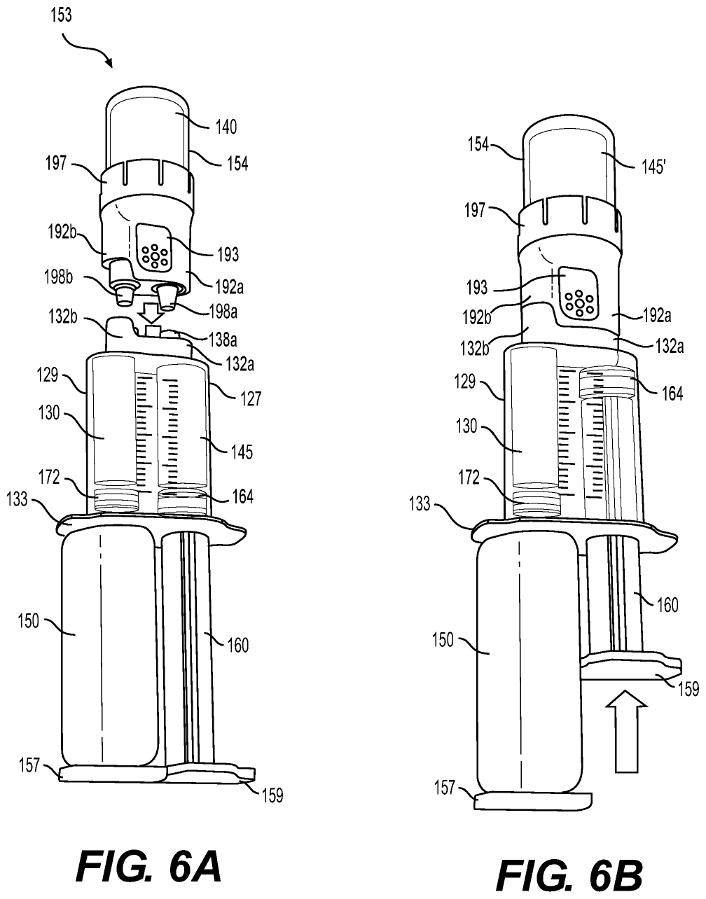

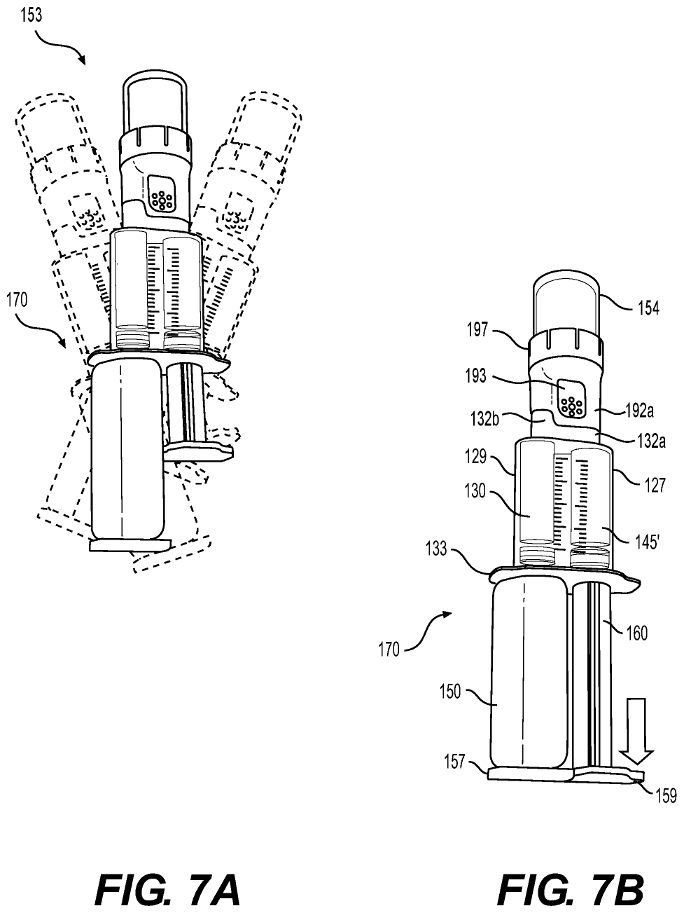

Lumen 127 can include one or more constituents (e.g., a fluid, liquid, powder, or some combination thereof). As used herein, the term “fluid” as it relates to constituents of system 100 is defined broadly and can include liquids, gels and particulate matter such as granules, pellets, or powders, or any combination of liquids, gels, oils, and/or particulate matter (e.g., granules, pellets, or powders). As desired, distally moving rod 160 can cause stopper 164 to advance fluid out from lumen 127 through port 138a, as shown in FIG. 4. In some examples, constituent(s) of lumen 127 can include constituent 145 (e.g., diluent). The diluent used in systems of this disclosure, including system 100, can be a branched polymer having a plurality of succinimidyl termini dissolved in a low pH (4.0) containing a low molecular weight precursor comprising nucleophiles, though other diluent fluid solutions are contemplated within the scope of this disclosure. Once constituents 140, 145 are mixed together, as shown between FIGS. 6A-7B, precursor solution 145′ can be formed in lumen 127.

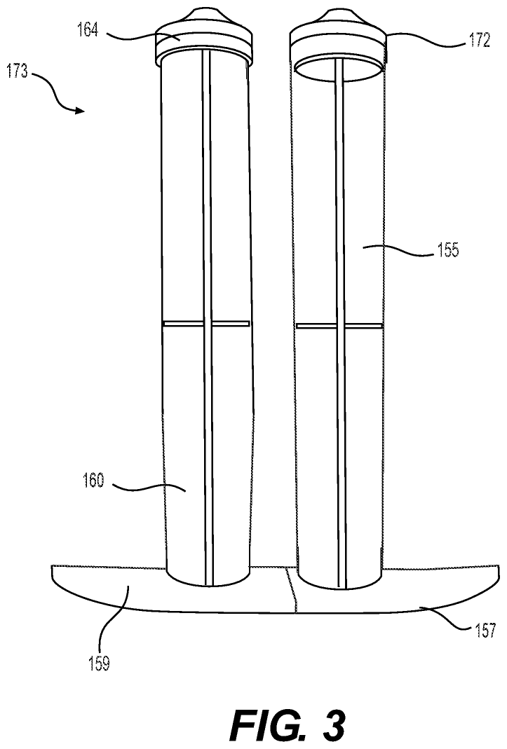

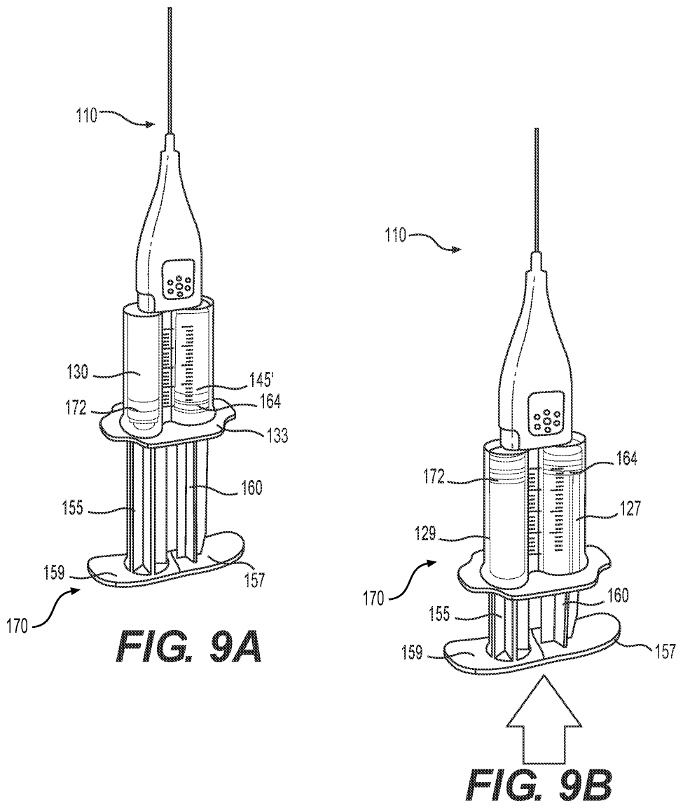

Lumen 129 can similarly include a plunger rod 155 slidable therein, as shown more clearly in FIGS. 3 and 9A. A distal end of rod 155 can include a stopper 172. A proximal end of rod 155 can include an actuating flange 157 configured so that a user can a press thereon to drive rod 155 proximally or distally. As seen in FIGS. 2-3, plunger rod 155, flange 157, rod 160, and lumen 127 can be partially or entirely integrally formed together to form plunger assembly 173.

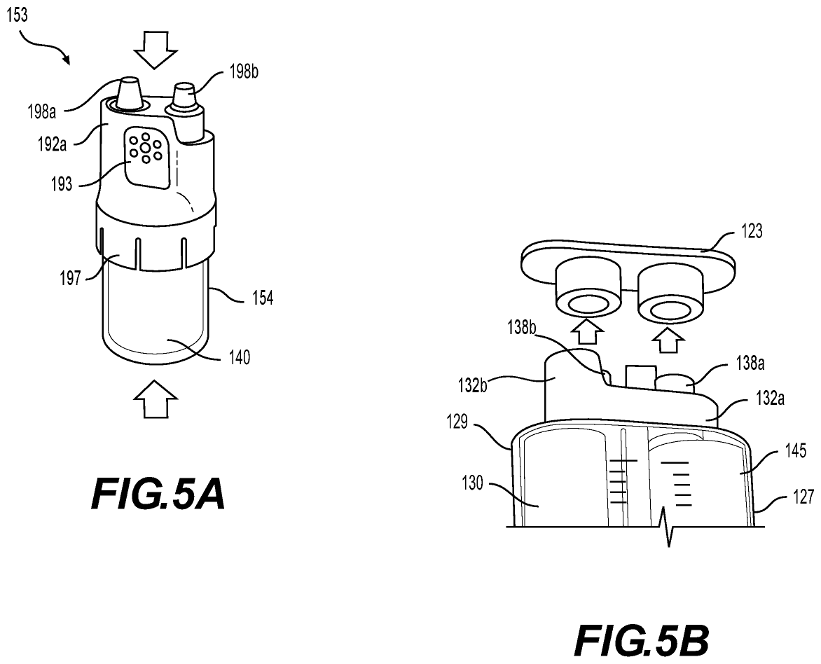

Each of ports 138a, 138b are configured to couple to corresponding receivers of connector 115 and permit egress of fluids from respective lumens 127, 129 into a mixing lumen of connector 115. The shape and position of ports 138a, 138b are clearly shown in FIG. 5B and FIG. 6A. In some aspects, port 138b can be positioned on one side of asymmetric coupler 132, for example portion 132b, which as shown in FIG. 5B and FIG. 6A can include shape that differs from portion 132a of coupler 132 opposite thereof. In this respect, portion 132a can be shaped different from portion 132b so that port 138a can only couple to a corresponding receiver side of connector 115. As can be seen, portion 115b of connector 115 is asymmetric with one side higher than the other and portions 132a, 132b of coupler 132 form a mirror shape of portion 115b. The shape of coupler 132 and portion 115b can be any shape so long as it is asymmetric to guide connector 115 and coupler 132 into the specific orientation and arrangement so that ports 138a, 138b are connectable in fluid communication with corresponding receivers (e.g., tubes 158, 162) of connector 115. In some aspects, stopper 172 of rod 155 can advance a constituent 130 (e.g., an accelerant) to mix with precursor 145′ once distal of ports 138a, b.

Referring to FIG. 2, vial adaptor 153 can include a vial 154 with contents for insertion from vial adaptor 153 into the main assembly 170. For example, the vial 154 can include constituent 140 (e.g., an activating agent such as a hydrophilic polymer, PEG, etc.) or some other constituent, such as constituent 145, for producing a precursor for a gel composition mixture of filler 30. Vial adaptor 153 can include an asymmetric coupler 192a, 192b with an outer profile substantially similar to the asymmetric coupler 132 of assembly 170. In this respect, the asymmetric shape of vial adaptor 153 is advantageously designed so as that adaptor 153 can only engage with assembly 170 in a specific orientation to ensure proper fluid coupling therebetween.

In a first state before mixing, the system 100 can include a retainer 150 removably positioned between flanges 133 and 157 so as to prevent unwanted movement rod of 155. In some aspects, constituent 130 can be positioned within lumen 129 (e.g., prepackaged or prefilled), as shown in FIG. 2. In some aspects, flange 157 can be permanently or temporarily attached to flange 159 of rod 160 so that distally advancing flange 157 also distally advances both stopper 172 as well as rod 160, and stopper 164. Precursor 145′ in lumen 127 and constituent 130 in lumen 129 can be capable of egressing through respective ports 138a, 138b and mixing together distal thereof (e.g., in the central lumen of connector 115). In some aspects, once flanges 157, 159 are aligned and/or otherwise attached, flange 157 being distally advanced can drive both rod 160 and rod 155 simultaneously.

Turning back to needle assembly 110, connector 115 can include a distal portion 115a and a proximal portion 115b with lower, asymmetric shape to couple to coupler 132. Portion 115b can be relatively solid, rather than relatively hollow, and insertable into an open proximal end of portion 115a to nest therewith and form connector 115. Portion 115a can be substantially hollow with a tapered or Y-shape profile for its outer surface. Portion 115a can terminate in a distal end with a central lumen running therethrough. Each of lumens 127, 129 can be in fluid communication with a proximal end of the central lumen of connector 115. Portion 115b can be asymmetric and coupled to coupler 132. The central lumen can include a static mixer so that fluid from respective lumens 127, 129 can mix together and form the gel composition to be delivered through needle 108.

In some aspects, portion 115b can include a tube (e.g., a hypotube) with a proximal end configured in fluid communication with lumen 127 and pierce a corresponding membrane or seal 136 of ports 138a, 138b. Portion 115b can also include a tube (e.g., a hypotube) with a proximal end configured in fluid communication with lumen 129 and pierce a corresponding membrane or seal 136 of port 138. In this respect, once precursors 145′ is in position in lumen 127 and constituent 130 is positioned in lumen 129 with air purged from each and connector 115 assembled thereto, distally moving rod 155 can cause precursor 145′ and constituent 130 to egress through respective ports 138 and respective tubes to mix with each other in the central lumen. The tubes can form a Y-shape, though any other shape can be used as needed or required.

Vial adaptor 153 can include a first fluid port 198b configured to removably connect with port 138b and a second alignment port 198a to removably connect with port 138a. Ports 198a, 198b can be surrounding by an outer profile of the asymmetric coupler 192a, 192b. A plurality of radially separated tabs 197 can extend from an outer surface of adaptor 153 that collectively form a flexible inner diameter to removably connect to an outer diameter of the vial 154. Each tab 197 can be separated by a notch or space. Each tab 197 can be biased inwardly so that an outer diameter of vial 154 can be larger than an inner diameter of collectively formed by tabs 197 but inserting vial 154 through an opening formed between the tabs 197 can cause tabs 197 to flex outwardly and frictionally attach to vial 154. In this respect, tabs 197 can collectively form a vial receiver.

Referring to FIG. 4 and FIG. 6A, the outer profile of the asymmetric coupler 132, as defined between its portions 132a, 132b, is shaped so that port 138a can only removably connect with port 198b and port 138b can only removably connect with port 198a. As shown in FIG. 2, portion 115b of connector 115 can include a similar or equivalent shape as coupler 192a, 192b of vial adaptor 153, which ensures correct alignment when coupling between assembly 170 and adaptor 153 or needle assembly 110. In some examples, port 198b can include an outer diameter that can be inserted into port 138b. Port 198b can include a rod, pin, dowel, or the like that plugs port 198b and prevent flow of any fluids therethrough. This is advantageous as it prevents unwanted flow between vial 154 and lumen 129 and ensures that the correct lumen (e.g., lumen 127) of assembly 170 receives contents from vial 154. In turn, mixing errors are minimized to the extent possible. For example, when assembly 170 is connected with vial adaptor 153, the respective asymmetric interfaces prevent incorrect alignment so fluids can only be exchanged between lumen 127 and vial 154 to form precursor 145′. In some examples, constituent 145 can be urged into vial 154 to mix with constituent 140 and then drawn back into lumen 127. However, in other examples constituent 145 can be packaged in vial 154 and constituent 140 can be first in lumen 127 so that constituent 145 is loaded from vial 154 through port 138a and into lumen 127. Regardless of location, contents of vial 154 can be loaded into lumen 127 to generate precursor 145′.

FIG. 4 shows an exemplary alignment and coupling between assembly 170 and vial adaptor 153. A tube 188 (e.g., a hypotube) can be in fluid communication to deliver contents between vial 154 and lumen 127. Tube 188 can include one or more bends or turns (e.g., one or more orthogonal turns) and be configured to run from port 198b to the interior of vial 154. Any unwanted air can be purged from vial 154 via exhaust path 187, as denoted by the dashed line in FIG. 4. In some examples, path 187 can include a vent (e.g., a one-way valve with an air-permeable fluid-impermeable membrane) for venting air in the tube 188, vial 154, and/or port 198b.

Now, turning to FIGS. 5A-9B are example steps of a process of using system 100 according to certain aspects of this disclosure. While certain steps are shown as a sequence between each figure, in other embodiments, fewer steps are contemplated and the order by which steps are performed can be different than what is illustrated. In FIG. 5A, vial adaptor 153 is introduced. In FIG. 5B system 100 is introduced and a cap 123 can be detached from ports 138a, 138b. Cap 123 can be configured to seal ports 138a, 138b between uses or during transit when stored in separate packaging or a kit. Lumen 127 can include constituent 145 (e.g., a diluent, a hydrophilic polymer, PEG, etc.,) whereas lumen 129 can include constituent 130 (e.g., accelerant).

In FIG. 6A, with cap 123 removed, adaptor 153 can be aligned and coupled to system via asymmetric couplers 132, 192. System 100 is illustrated in a first state with rod 155 fully retracted and retainer 150 positioned between flanges 133, 157. In the depicted configuration, rod 155 is incapable of distally moving as a result of retainer 150 being wedged between flanges 133, 157. In contrast, precursor 145′ is capable of being mixed by the depicted coupling of vial adaptor 153 with assembly 170. The asymmetric shape of coupler 132 ensures proper coupling between ports 138a, 138b and ports 198a, 198b. In FIG. 6A specifically, rod 160 is fully withdrawn and capable of advancing constituent 145 from lumen 127 through portion 132a and port 198a ultimately into vial 154. This is more clearly shown in FIG. 6B where rod 160 has been advanced causing stopper 164 inject constituent 145 into vial 154. Accordingly, in the depicted first state of FIGS. 6A and 6B, coupler 132 is removably connected with the asymmetric coupler 192a, 192b so that moving rod 160 causes contents of vial 154 (e.g., constituent 145) to be delivered from port 138a and into vial 154 via port 198a, to facilitate formation of a mixture (e.g., precursor 145′).

Now, constituents 140, 145 can mix together and form precursor 145′. For example, assembly 170 and vial adaptor 153 can be shaken back and forth as in FIG. 7A to ensure precursor 145′ forms as a result of mixing between constituents 140, 145, while constituent 130 remains in lumen 129. Preferably, the shaking action of FIG. 7A is done while the ports 138a, 138b are oriented generally upward. While vial adaptor 153 is illustrated during the act of shaking in FIG. 7A so that precursor 145′ can be mixed inside vial 154, in other examples vial adaptor 153 can be detached from assembly 170 prior to shaking. Further, the shaking to effect proper mixing of precursor 145′ can be performed in other orientations (e.g., generally downward, etc.), as needed or required.

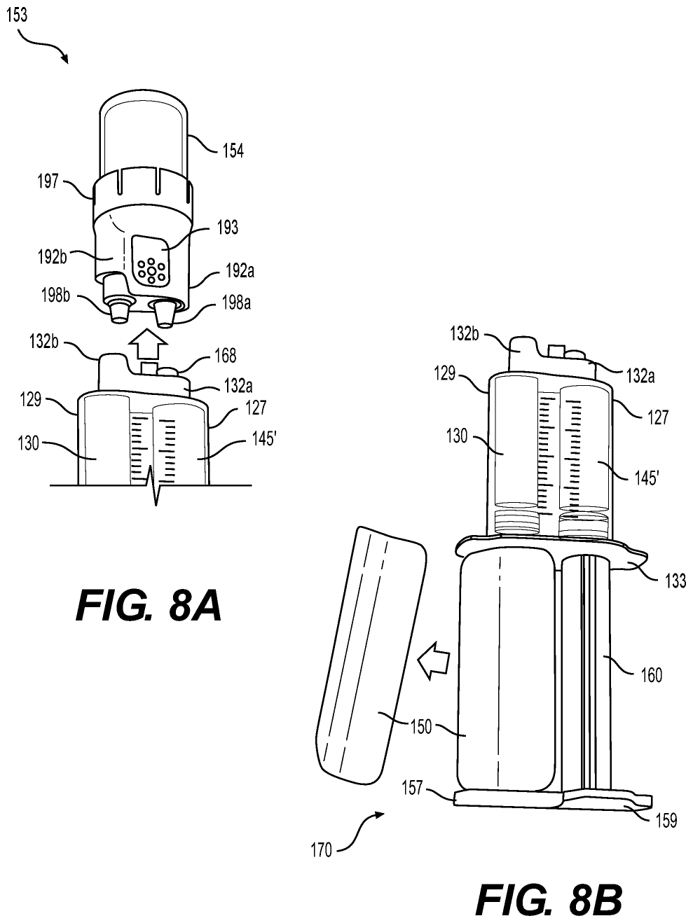

In FIG. 7B, a second state is illustrated with rod 160 being proximally moved, as denoted by the drawn arrow, so that precursor 145′ is drawn from vial 154 to lumen 127. Flange 159 can be advanced until aligned or attached with flange 157. In some examples, once flange 159 is so aligned or attached with flange 157, this can indicate that lumen 127 has all precursor 145′ from vial 154. With precursor 145′ formed and present in lumen 127 and constituent 130 in lumen 129, vial adaptor 153 can be removed from assembly 170, as in FIG. 8A. In some examples, vial adaptor 153 can include an externally positioned button 193 to open and close corresponding connecting latches of connector vial adaptor 153. In this respect, adaptor 153 can include one or more latches configured to removably attach to coupler 132 via one or more latches controlled by button 193. With vial adaptor 153 disconnected from assembly 170, in FIG. 8B retainer 150 can be removed. While not shown, flange 157 can be distally advanced slightly to purge any air from system 100 out through respective ports 138a, 138b.

In FIG. 9A, system 100 is now connected to connector 115, which is primed and connected to needle 108 in position at the treatment site. Aspects of priming connector 115 are discussed more in FIGS. 10A-10C. In FIG. 9B, a user can advance flange 157 distally, as denoted by the upward arrow, so that corresponding rods 155, 160 distally drive respective stoppers 164, 172 and advance precursor 145′ from lumen 127 and constituent 130 from lumen 129, through ports 138a, 138b, and into the central lumen of connector 115. In some examples, as long as flange 157 continues advancing, precursor 145′ and constituent 130 can mix within the central lumen and continue egressing through needle 108 and ultimately to the treatment site. Optionally, the central lumen can include a static mixer configured to thoroughly mix the constituents together to form the gel composition to be delivered to the treatment site. System 100 as shown is relatively easy to assemble and minimizes potential unintentional gel mixing errors prior to delivery.

Separately, in FIG. 10A, connector 115 is shown connected to syringe 200 via adaptor 120, as illustrated more clearly in Fig. w which is a close-up of section A. While not shown, during use connector 115 is contemplated to be connected to needle 108 for hydrodissection at the treatment site with saline from the syringe 200, prior to connector 115 being assembled with main assembly 170. Adaptor 120 can provide a fluid bridge 122 from each of tubes 158, 162 of connector 115 to syringe 200. Connector 115 can include an externally positioned button 113 to open and close corresponding connecting latches of connector 115. In this respect, adaptor 120 can include one or more latches 139 configured to removably attach to connector 115 via button 113. Adaptor 120 can couple to syringe 200 with a luer fitting (e.g., end 121) or any other connector operable to connect with a distal end of syringe 200. After hydrodissection with syringe 200, adaptor 120 can be released from connector 115, as shown in FIG. 10C, and then coupled with assembly 170. However, other coupling approaches between connector 115, adaptor 120, and syringe 200 are contemplated as needed or required. For example and without limitation, snap fit connectors, magnetic connectors, female-male connectors, hook and loop fasteners and the like are contemplated

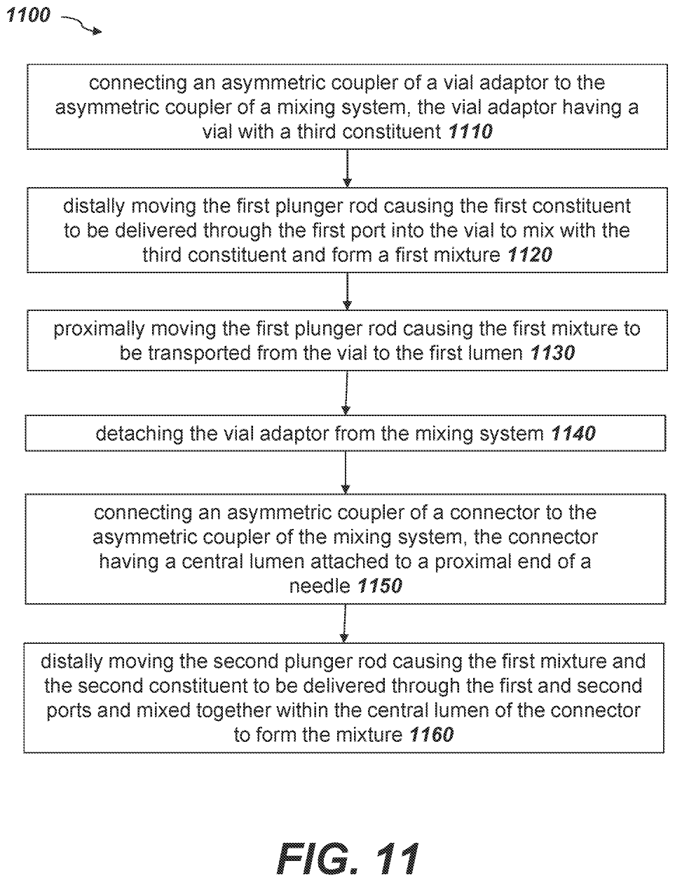

FIG. 11 depicts a method 1100 of using any of the herein disclosed mixing systems. Step 1110 of method 1100 can include connecting an asymmetric coupler of a vial adaptor to the asymmetric coupler of a mixing system, the vial adaptor having a vial with a third constituent. Step 1120 of method 1100 can include distally moving the first plunger rod causing the first constituent to be delivered through the first port into the vial to mix with the third constituent and form a first mixture. Step 1130 of method 1100 can include proximally moving the first plunger rod causing the first mixture to be transported from the vial to the first lumen. Step 1140 of method 1100 can include detaching the vial adaptor from the mixing system. Step 1150 of method 1100 can include connecting an asymmetric coupler of a connector to the asymmetric coupler of the mixing system, the connector having a central lumen attached to a proximal end of a needle. Step 1160 of method 1100 can include distally moving the second plunger rod causing the first mixture and the second constituent to be delivered through the first and second ports and mixed together within the central lumen of the connector to form the mixture. Method 1100 can end after step 1160. In other embodiments, additional or fewer steps according to the examples described above can be performed.

Other aspects and embodiments of the present disclosure will be apparent to those skilled in the art from consideration of the specification and practice of the embodiments disclosed herein. While certain features of the present disclosure are discussed within the context of exemplary procedures, the compositions, systems, and methods may be used for other medical procedures according to the general principles disclosed. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein. It is intended that the specification and examples be considered as exemplary only, with a true scope and spirit of the present disclosure being indicated by the following claims.

What is claimed is:

1. A system for producing a mixture to deliver to a treatment site, comprising:
a mixing lumen attachable to a proximal end of a delivery system;
a multi-lumen chamber removably connected to a proximal end of the mixing lumen, the multi-lumen chamber comprising a first lumen aligned with a second lumen;
the first lumen configured to comprise a first constituent in a first state, the first lumen comprising a first plunger to move the first constituent from the first lumen to the mixing lumen, and the first lumen terminating in a first port;
the second lumen configured to comprise a second constituent, the second lumen comprising a second plunger to distally move the second constituent, and the second lumen terminating in a second port; and
a vial adaptor comprising a vial with a third constituent, the vial adaptor configured to connect to the multi-lumen chamber.

2. The system of claim 1, wherein the first and second lumens are adjacent each other.

3. The system of claim 1, the second lumen comprising a second plunger rod internally positioned therein to distally move the second constituent and the first constituent.

4. The system of claim 3, wherein in a third state, distally moving the second plunger rod causes a first mixture and the second constituent to be delivered through the first and second ports, mixed together within the mixing lumen to form the mixture, and delivered through the delivery system.

5. The system of claim 1, wherein in a second state, proximally moving a first plunger rod of the first lumen causes a first mixture to be transported from the vial to the first lumen.

6. The system of claim 1, wherein the mixing lumen is in a connector comprising a first asymmetric coupler positioned on a proximal end of the connector.

7. The system of claim 6, wherein the multi-lumen chamber comprises a second asymmetric coupler positioned on a distal end of the multi-lumen chamber and configured to removably connect to the first asymmetric coupler.

8. The system of claim 7, wherein the vial adaptor comprises an asymmetric coupler comprising an outer profile substantially similar to the first asymmetric coupler so as to removably connect with the second asymmetric coupler.

9. The system of claim 8, wherein in the first state, the second asymmetric coupler is connected with the asymmetric coupler of the vial adaptor so that distally moving a first plunger rod of the first lumen causes the first constituent to be delivered through the first port into the vial to form a first mixture.

10. The system of claim 8, wherein the vial adaptor further comprises a first fluid port to removably connect with the first port and a second alignment port to removably connect with the second port, the first fluid port and the second alignment port being surrounding by the outer profile of the asymmetric coupler.

11. The system of claim 10, the vial adaptor further comprising:
a tube between the vial and the first fluid port; and
a vent for venting air in the tube, the vial, and the first fluid port, the vent being positioned between the vial and the first fluid port.

12. The system of claim 11, wherein the tube comprises one or more approximately perpendicular bends distal of the vial and proximal of the first fluid port; and
wherein the vent is positioned proximal or adjacent the one or more approximately perpendicular bends.

13. A method for producing a mixture with a mixing system to deliver to a treatment site, the mixing system comprising a multi-lumen chamber comprising a first lumen aligned with a second lumen and an asymmetric coupler on a distal end of the multi-lumen chamber;
the first lumen comprising a first constituent and a first plunger rod internally positioned to move one or more constituents from the first lumen through a first port positioned at a distal end; and
the second lumen comprising a second constituent and a second plunger rod internally positioned to move one or more constituents from the first and second lumens lumen through the first port and a second port positioned at a distal end of the second lumen, the method comprising
connecting an asymmetric coupler of a vial adaptor to the asymmetric coupler of a mixing system, the vial adaptor comprising a vial with a third constituent;
distally moving the first plunger rod causing the first constituent to be delivered through the first port into the vial to mix with the third constituent and form a first mixture;
proximally moving the first plunger rod causing the first mixture to be transported from the vial to the first lumen;
detaching the vial adaptor from the mixing system;
connecting an asymmetric coupler of a connector to the asymmetric coupler of the mixing system, the connector comprising a central lumen attached to a proximal end of a needle; and
distally moving the second plunger rod causing the first mixture and the second constituent to be delivered through the first and second ports and mixed together within the central lumen of the connector to form the mixture.

14. The method of claim 13, wherein the vial adaptor further comprises a first fluid port to removably connect with the first port and a second alignment port to removably connect with the second port, the first fluid port and the second alignment port being surrounding by an outer profile of the asymmetric coupler.

15. The method of claim 13, wherein the vial adaptor further comprises a first fluid port to removably connect with the first port and a second alignment port to removably connect with the second port, wherein an outer profile of the asymmetric coupler is shaped so that the first port can only removably connect with the first fluid port and the second port can only removably connect with the second alignment port.

16. The method of claim 13, wherein the vial adaptor further comprises a first fluid port to removably connect with the first port and a second alignment port to removably connect with the second port, the second alignment port comprising an outer diameter configured to be inserted into the second port, the second alignment port comprising a dowel configured to plug the second port and prevent flow of the second constituent.

17. The method of claim 13, wherein the vial adaptor further comprises:
a first fluid port to removably connect with the first port and a second alignment port to removably connect with the second port;
a tube between the vial and the first fluid port; and
a vent for venting air in the tube, the vial, and the first fluid port, the vent being positioned between the vial and the first fluid port.

18. The method of claim 17, wherein the tube comprises one or more approximately perpendicular bends distal of the vial and proximal of the first fluid port; and
wherein the vent is positioned proximal or adjacent the one or more approximately perpendicular bends.

19. The method of claim 13, further comprising: purging air from the first and second lumens by distally moving the second plunger rod a first distance prior to forming the mixture.

20. The method of claim 13, wherein the connector is a Y-shaped connector, the method further comprising:
positioning the asymmetric coupler at or adjacent the connector;
positioning a first tube in fluid communication with the first fluid port and the central lumen of the connector; and
positioning a second tube in fluid communication with the second port and the central lumen of the connector.
